(19) United States
(12) Patent Application Publication
(10) Pub. No.: US 2023/0302229 A1
(43) Pub. Date: Sep. 28, 2023

(54) VOICE-CONTROLLED DRUG INJECTOR

(71) Applicant: ZHEJIANG UNIVERSITY, Hangzhou (CN)

(72) Inventors: Xin YU, Hangzhou (CN); Hongyuan LV, Hangzhou (CN); Hong YU, Hangzhou (CN)

(73) Assignee: ZHEJIANG UNIVERSITY, Hangzhou (CN)

(21) Appl. No.: 18/069,362

(22) Filed: Dec. 21, 2022

(30) Foreign Application Priority Data
Mar. 22, 2022 (CN) 202210284672 .4

(51) Int. Cl. A61M 5/315; A61M 5/20

(52) U.S. Cl. CPC A61M 5/3158; A61M 5/204; A61M 2205/80; A61M 2205/3327

(57) ABSTRACT

Disclosed is a voice-controlled drug injector, comprising an extension tube, a voice control receiver, and an injection pusher. One end of the extension tube is a liquid drug outlet and is connected to a needle, and the other end of the extension tube is a liquid drug inlet and is connected to one end of the injection pusher so as to form a drug flowing channel. The voice control receiver is connected to the other end of the injection pusher and is configured to receive a voice control instruction to control the injection pusher to perform drug injection. In accordance with the present disclosure, the pumpback and drug injection work requiring the cooperation of two people during conventional neural blockade operation can be completed by means of voice control of a single person, the labor consumption is greatly reduced, the operation time is saved, the neural block efficiency is improved.

### CROSS-REFERENCE TO RELATED APPLICATION

This patent application claims the benefit and priority of Chinese Patent Application No. 202210284672.4, filed with the China National Intellectual Property Administration on Mar. 22, 2022, the disclosure of which is incorporated by reference herein in its entirety as part of the present application.

### TECHNICAL FIELD

The present disclosure relates to the field of medical devices, and specifically a voice-controlled drug injector.

### BACKGROUND

Ultrasound-guided neural blockade is the most clinically significant technique in the field of regional anesthesia in recent years. With the help of the ultrasound which is the third eye, many superficial and deep nerve structures can be clearly displayed, and puncturing around the target nerve using a block needle can be achieved under real-time, dynamic ultrasound guidance to accurately perform neural blockade, thus unnecessary nerve damage is avoided, the incidence of complications is reduced, and the effectiveness of the neural blockade is improved. With the increasing maturity and popularity of such technique, ultrasound-guided neural blockade has become a routine practice for anesthesiologists. Such operation requires the anesthesiologist to hold the ultrasound probe with one hand while insert the needle with the other hand, and requires an assistant to perform the pumpback and drug bolus, such process requires close cooperation between the anesthesiologist and the assistant so as to obtain a consistent judgment on the timing and dose of drug injection, the manpower waste is caused, and the poor cooperation may influence the effect of neural blockade. Although neural blockade devices capable of being operated by one hand are available at present, which can reduce labor consumption, the operator still needs complete both the tasks of fixing the block needle and injecting the drug at the same time with one hand, this may easily cause the deviation of the block needle's position or the injection dose during the operation. Moreover, the ultrasound-guided injection will be carried out in multiple sites, involving multiple complicated procedure. However, the single button lacks accurate control, so how to complete the ultrasound-guided neural blockade independently and accurately is still a problem to be solved.

### SUMMARY

To this end, the present disclosure provides a voice-controlled drug injector. In accordance with the present disclosure, the pumpback and drug injection work requiring the cooperation of two people during conventional neural blockade operation can be completed through voice control of a single person, thus the labor consumption is greatly reduced and the neural blockade efficiency is improved.

The present disclosure provides a voice-controlled drug injector. The voice-controlled drug injector comprises an extension tube, a voice control receiver, and an injection pusher. One end of the extension tube is a liquid drug outlet which is connected to a needle, and the other end of the extension tube is a liquid drug inlet which is attached to the syringe nipple of the injection pusher to form a drug flowing channel. The voice control receiver is connected to the other end of the injection pusher and is configured to receive a voice control instruction so as to control the injection pusher to perform drug injection.

Further, the voice-controlled drug injector further comprises a bottom plate, the injection pusher and the voice control receiver are both arranged on the bottom plate.

Further, the injection pusher comprises a syringe, and a propulsion device for driving the syringe. The nipple of the syringe is connected to the liquid drug inlet, and the tail end of the syringe core rod is connected to the propulsion device.

Further, the injection pusher further comprises a pressing handle for fixing the syringe. The propulsion device comprises a push block connected to the core rod and configured to push the core rod to move, and a fixed seat connected to the push block. The pressing handle is arranged at one end of the bottom plate, the fixed seat is arranged at the other end of the bottom plate, and the push block as well as the syringe are located between the pressing handle and the fixed seat.

Further, the propulsion device further comprises a guide rod for stabilizing a moving track, and a lead screw capable of reciprocating. The guide rod is arranged between the push block and the fixed seat, and the lead screw is arranged inside the guide rod and is connected to a driving device in the fixed seat.

Further, the fixed seat is internally provided with a power supply with a USB interface, and an encoder for monitoring the number of revolutions of the driving device.

Further, the pressing handle is provided with a clamping groove, and the syringe is clamped into the clamping groove for fixation.

Further, the push block is provided with a displacement sensor for calculating the amount of movement of the syringe core rod, a converter for calculating injection dose according to the amount of movement, and a voice broadcaster for real-time broadcasting, and the converter is in signal connection with the displacement sensor and the voice broadcaster.

Further, the voice-controlled drug injector further comprises a negative pressure airbag and a connecting tube. One end of the connecting tube is connected to a sidewall of the extension tube, the other end of the connecting tube is connected to the negative pressure airbag, and a connecting part of the connecting tube and the extension tube is provided with a check valve for preventing the liquid drug from entering the negative pressure airbag.

Further, the syringe is detachably connected to the propulsion device, and the syringe is externally provided with scales for observing the bolus dose of the liquid drug.

Compared with the prior art, the present disclosure has the following beneficial effects:

The voice-controlled drug injector provided by the present disclosure is scientific and reasonable in structure, safe and convenient to operate and user-friendly in design. Through the design of the voice control receiver and the injection pusher, the pumpback and drug injection work requiring the cooperation of two people during conventional neural blockade operation can be completed by means of voice control of a single person, the labor consumption is greatly reduced, the operation time is saved, and the neural blockade efficiency is improved. The voice-controlled drug injector has more accurate block effect and has broad clinical perspectives.

### BRIEF DESCRIPTION OF THE DRAWINGS

FIG. 1 is a structure diagram of a voice-controlled drug injector in accordance with the present disclosure.

In the drawings: 10—extension tube; 11—liquid drug outlet; 12—liquid drug inlet; 20—voice control receiver; 30—injection pusher; 31—syringe; 311—core rod; 312—scale; 32—propulsion device; 321—push block; 3211—displacement sensor; 3212—converter; 3213—voice broadcaster; 322—guide rod; 323—lead screw; 324—fixed seat; 3241—USB interface; 33—pressing handle; 331—clamping groove; 40—negative pressure airbag; 41—exhaust hole; 42—bottle cap; 421—opening; 50—connecting tube; 60—bottom plate.

### DETAILED DESCRIPTION OF THE EMBODIMENTS

The specific embodiments of the present disclosure are further described in detail below with reference to the accompanying drawings and embodiments. The following embodiments are configured to illustrate the present disclosure rather than limiting the scope of the present disclosure.

In the description of the present disclosure, it needs to be noted that orientation or positional relationship indicated in the description is based on the orientation or positional relationship shown in the drawings only for convenience of description of the present disclosure and simplification of description rather than indicating or implying that the device or element referred to must have a particular orientation, be constructed and operate in a particular orientation, and thus are not to be construed as limiting the present disclosure.

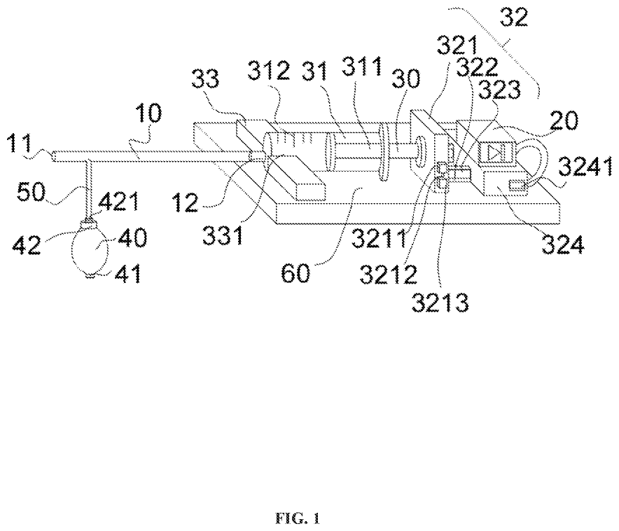

Please referring to FIG. 1, the present disclosure provides a voice-controlled drug injector. The voice-controlled drug injector comprises an extension tube 10, a voice control receiver 20, an injection pusher 30, a negative pressure airbag 40, a connecting tube 50, and a bottom plate 60. One end of the extension tube 10 is a liquid drug outlet 11 and is connected to a needle, the other end of the extension tube is a liquid drug inlet 12 and is connected to one end of the injection pusher 30. One end of the connecting tube 50 is connected to the negative pressure airbag 40, the other end of the connecting tube 50 is connected to a sidewall of the extension tube 10. The voice control receiver 20 is connected to the injection pusher 30, and the voice control receiver 20 and the injection pusher 30 are both located on the bottom plate 60, and the voice control receiver 20 is configured to receive a voice control instruction so as to control the injection pusher 30 to perform drug injection.

Specifically, the extension tube 10 is a hollow tubular structure and is connected to the injection pusher 30 to construct a drug outflow channel. An opening is formed in the sidewall of a tube cavity and is connected to one end of the connecting tube 50. The tube wall of the extension tube 10 is transparent, thus facilitating the observation of the color of the pumpback liquid and determining whether there is blood reflux (whether the block needle is located in the blood vessel).

Specifically, the negative pressure airbag 40 is an elliptical structure, and is configured to provide a negative pressure for sucking the liquid drug. The lower end of the negative pressure airbag 40 is provided with an exhaust hole 41, and the upper end of the negative pressure airbag is provided with a columnar bottle cap 42. The top end of the bottle cap 42 is provided with an opening 421, and the opening 421 is connected to the other end of the connecting tube 50. The connecting part between the connecting tube 50 and the sidewall of the extension tube 10 is provided with a check valve for preventing the liquid drug from entering the negative pressure airbag 40. The check valve is covered with a thin film and thus is not shown in the FIGURE.

Specifically, the injection pusher 30 comprises a syringe 31, a propulsion device 32 for driving the syringe 31, and a pressing handle 33 for fixing the syringe, the pressing handle 33 is arranged at one end of the bottom plate 60, and the propulsion device 32 is arranged at the other end of the bottom plate 60.

Further, the syringe 31 is detachably connected to the propulsion device 32, and thus the syringe 31 may be dismounted from the injection pusher 30 for liquid drug supplementing. The nipple of the syringe 31 is connected to the liquid drug inlet 12 of the extension tube 10, and the tail end of the syringe core rod 311 is connected to the propulsion device 32. A cavity wall of the syringe 31 is transparent, and the outside thereof is marked with scales 312, such as the volume of 10, 20, 50 ml, etc., thus the bolus dose of the liquid drug can be observed from the outside.

Specifically, the pressing handle 33 is arranged at one end, close to the extension tube 10, of the syringe 31, a clamping groove 331 is arranged on the pressing handle 33, and the syringe 31 is clamped into the clamping groove 331. When the propulsion device 32 pushes the core rod 311, one end, away from the core rod 311, of the syringe 31 can be fixed by the pressing handle 33 and cannot move along with the movement of the core rod 311.

Specifically, the propulsion device 32 comprises a push block 321 for fixing the core rod 311, a guide rod 322 for stabilizing a moving track, a lead screw 323 capable of reciprocating, and a fixed seat 324. The guide rod 322 is arranged between the push block 321 and the fixed seat 324, the lead screw 323 is arranged in the guide rod 322, and voice control receiver 20 is arranged at the upper part of the fixed seat 324. It needs to be noted that the guide rod 322 in the present disclosure is elastic and telescopic. The fixed seat 324 is internally provided with a driving device connected to the lead screw 323 and configured to drive the lead screw 323 to move, a power supply with a USB interface 3241, and an encoder for monitoring the number of revolutions of the driving device. The push block 321 is provided with a displacement sensor 3211 for calculating the amount of movement of the syringe core rod 311, a converter 3212 for calculating the dose of injection according to the amount of movement, and a voice broadcaster 3213 for real-time broadcasting. The converter 3212 is arranged between the displacement sensor 3211 and the voice broadcaster 3213. The displacement sensor 3211, the converter 3212, the voice broadcaster 3213 and the voice control receiver 20 are in signal connection.

Specifically, the voice control receiver 20 is a voice-controlled switch and is internally provided with a circuit module for performing micro processing on signals fed back by the voice control receiver 20. The voice control receiver 20 is installed at the upper part of the fixed seat 324 in the injection pusher 30 to receive a voice control instruction (command words push/stop) for turning on or turning off the pusher 30.

A working method of a voice-controlled drug injector is as follows:
(1) A liquid drug outlet of an extension tube 10 is connected to a block needle.
(2) An ultrasonic probe is held with one hand for nerve localization, and the needle is inserted by the other hand to fix the block needle around the ultrasound-localized nerve.
(3) A negative pressure airbag 40 is pressed for negative pressure suction;
if there is no blood reflux in the extension tube 10, after calling “push” towards a voice control receiver 20, an injection pusher 30 starts drug bolus, a converter 3212 calculates the dose of injection according to a displacement sensor 3111, and a voice broadcaster 3212 broadcasts the injection volume, counting off per ml. After the target dose is achieved, “stop” is called towards the voice control receiver 20, and then the injection pusher 30 stops drug bolus;
if there is blood reflux in the extension tube 10, the operation is stopped, and the ultrasonic probe is configured to localize nerves again, the step (3) is repeated, and the reflux blood is injected into the human body together with the liquid drug bolus.
(4) After the position of the block needle is changed, the steps (1) to (3) are repeated for operation.

It can be known from the above that voice-controlled drug injector provided by the present disclosure is scientific and reasonable in structure, safe and convenient to operate and user-friendly in design. Through the design of the voice control receiver and the injection pusher, the pumpback and drug injection work requiring the cooperation of two people during conventional neural blockade operation can be completed by means of voice control of a single person, the labor consumption is greatly reduced, the operation time is saved, and the neural block efficiency is improved. The voice-controlled drug injector has more accurate block effect and has broad clinical perspectives

The foregoing are merely specific embodiments of the present disclosure but are not intended to limit the scope of protection of the present disclosure. Any variation or replacement readily figured out by those skilled in the art within the technical scope disclosed in the present disclosure shall fall within the scope of protection of the present disclosure. Therefore, the scope of protection of the present disclosure shall be subject to the scope of protection of the claims.

We claim:

1. A voice-controlled drug injector, comprising an extension tube (10), a voice control receiver (20), and an injection pusher (30), wherein one end of the extension tube (10) is a liquid drug outlet (11) and is connected to a needle, the other end of the extension tube is a liquid drug inlet (12) and is connected to one end of the injection pusher (30) to form a drug flowing channel, and the voice control receiver (20) is connected to the other end of the injection pusher (30) and is configured to receive a voice control instruction so as to control the injection pusher (30) to perform drug injection.

2. The voice-controlled drug injector according to claim 1, further comprising a bottom plate (60), wherein the injection pusher (30) and the voice control receiver (20) are both arranged on the bottom plate (60).

3. The voice-controlled drug injector according to claim 2, wherein the injection pusher (30) comprises a syringe (31), and a propulsion device (32) for driving the syringe (31); one end of the syringe (31) is connected to the liquid drug inlet (12), and the other end of the syringe is connected to the propulsion device (32) by means of a core rod (311).

4. The voice-controlled drug injector according to claim 3, wherein the injection pusher (30) further comprises a pressing handle (33) for fixing the syringe (31), the propulsion device (32) comprises a push block (321) connected to the core rod (311) and configured to push the core rod (311) to move, and a fixed seat (324) connected to the push block (321); the pressing handle (31) is arranged at one end of the bottom plate (60), the fixe seat (324) is arranged at the other end of the bottom plate (60), and the push block (321) and the syringe (31) are located between the pressing handle (33) and the fixed seat (324).

5. The voice-controlled drug injector according to claim 4, wherein the propulsion device (32) further comprises a guide rod (322) for stabilizing a moving track, and a lead screw (323) capable of reciprocating; the guide rod (322) is arranged between the push block (321) and the fixed seat (324), and the lead screw (323) is arranged inside the guide rod (322) and is connected to a driving device in the fixed seat (324).

6. The voice-controlled drug injector according to claim 4, wherein the fixed seat (60) is internally provided with a power supply with a USB interface (3241), and an encoder for monitoring the number of revolutions of the driving device.

7. The voice-controlled drug injector according to claim 4, wherein the pressing handle (33) is provided with a clamping groove (331), and the syringe (31) is clamped into the clamping groove (331) for fixation.

8. The voice-controlled drug injector according to claim 4, wherein the push block (321) is provided with a displacement sensor (3211) for calculating the amount of movement of the syringe (31), a converter (3212) for calculating injection dose according to the amount of movement, and a voice broadcaster (3213) for real-time broadcasting, and the converter (3212) is in signal connection with the displacement sensor (3211) and the voice broadcaster (3213).

9. The voice-controlled drug injector according to claim 1, further comprising a negative pressure airbag (40) and a connecting tube (50), wherein one end of the connecting tube (50) is connected to a sidewall of the extension tube (10), the other end of the connecting tube is connected to the negative pressure airbag (40), and a connecting part of the connecting tube (50) and the extension tube (10) is provided with a check valve for preventing the liquid drug from entering the negative pressure airbag (40).

10. The voice-controlled drug injector according to claim 3, wherein the syringe (31) is detachably connected to the propulsion device (32), and the syringe (31) is externally provided with scales (312) for observing the bolus dose of the liquid drug.
